(19) United States
(12) Patent Application Publication
(10) Pub. No.: US 2025/0352392 A1
(43) Pub. Date: Nov. 20, 2025

(54) DEVICES AND METHODS FOR PROVIDING CONTROL DATA FOR AN OPHTHALMOLOGICAL LASER OF A TREATMENT APPARATUS FOR REDUCING GEOMETRIC IRREGULARITIES OF AN EYE

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 19/169,222

(22) Filed: Apr. 3, 2025

(30) Foreign Application Priority Data
May 16, 2024 (DE) 10 2024 113 794.0

(51) Int. Cl. A61F 9/008; G16H 20/40

(52) U.S. Cl. CPC A61F 9/008; G16H 20/40; A61F 2009/00848; A61F 2009/00872

(57) ABSTRACT

The invention relates to devices and methods for providing control data for an ophthalmological laser (12) of a treatment apparatus (10) for reducing geometric irregularities (14) of an eye. As steps, the method includes determining geometric irregularities (14) of the eye from predetermined examination data, which generate higher order aberrations; determining a treatment profile with a preset optical zone depending on the geometric irregularities (14), wherein an optimization function, which includes a term for reducing the higher order aberrations and an opposing tissue removal term, is optimized up to an optimization range for determining the treatment profile; and providing the control data, which includes at least the treatment profile.

### FIELD

The invention relates to a method for providing control data for an ophthalmological laser of a treatment apparatus for reducing geometric irregularities of an eye. Furthermore, the invention relates to a control device, which is configured to perform the method, to a treatment apparatus with such a control device as well as to a computer program including commands, which cause the treatment apparatus to perform the method, and to a computer-readable medium, on which the computer program is stored.

### BACKGROUND

Treatment apparatuses and methods for controlling ophthalmological lasers for the correction of an optical visual disorder and/or of pathologically or unnaturally altered areas of the cornea are known in the prior art. Therein, pulsed lasers and a beam focusing device may for example be formed such that laser pulses effect a photodisruption and/or ablation in a focus situated within the organic tissue to remove a tissue, in particular a tissue lenticule, from the cornea.

If the eye, in particular the cornea, has geometric irregularities, vision improvement by changing the corneal curvature is difficult. Geometric irregularities may for example be elevations and/or thinning in the eye, in particular the cornea, which may for example arise by a keratoconus, a keratoglobus, a pellucid marginal degeneration, an inflammation in the eye and/or by scars in the eye, for example by improper treatments. Thus, the cornea may for example be no longer smooth, but have one or more elevations or depressions, which generate aberrations and thus disturb a vision. This means that the geometric irregularities are not conventional impairments of vision like myopia or hyperopia. Therefore, the geometric irregularities cannot be improved by spectacle correction, in particular by a laser treatment, in which values for sphere, cylinder and axis are adapted.

### SUMMARY

It is the object of the present invention to treat geometric irregularities of an eye in an improved manner.

This object is solved by the independent claims. Advantageous developments of the invention are disclosed in the dependent claims, in the following description as well as the figures.

The invention is based on the idea that a treatment is planned, by which the geometric irregularities are cut or ablated such that vision impairing effects are minimized and as much corneal tissue as possible is additionally preserved.

An aspect of the invention relates to a method for providing control data for an ophthalmological laser of a treatment apparatus for reducing geometric irregularities of an eye, wherein the method comprises the following steps performed by a control device. Therein, an appliance or an appliance component is to be understood by a control device, which for example comprises a processor or microprocessor, wherein the following steps may be automatically or semi-automatically performed by the control device.

Determining geometric irregularities of the eye from predetermined examination data, which generate higher order aberrations, determining a treatment profile with a preset optical zone depending on the geometric irregularities, wherein an optimization function, which includes a term for reducing the higher order aberrations and an opposing tissue removal term, is optimized up to an optimization range for determining the treatment profile, and providing the control data, which includes at least the treatment profile, are effected.

In other words, geometric irregularities may be present in the eye or in the cornea. For example, the geometric irregularities may be elevations and/or depressions, by which higher order aberrations are generated. The geometric irregularities or the higher order aberrations may for example be predetermined by examinations and be provided to the control device as examination data. Herein, a corneal profile may in particular be determined, for example by a topography measurement, or wavefront profiles of the eye may be ascertained. Thus, the geometric irregularities may be depicted as an optical error.

For compensating for or reducing the geometric irregularities, a treatment profile may be planned, wherein an optical zone is for example preset as a specification of a treatment area, in which higher and lower order aberrations may be varied for the treatment planning. In particular, it may be provided that an optimization function is optimized with optimization calculation approaches, wherein the optimization function includes a term for reducing the higher order aberrations and an opposing tissue removal term. Herein, the higher order aberrations to be corrected may in particular be varied between the originally determined value and zero, wherein an individual reduction may be determined for each higher order aberration or globally for all higher order aberrations. For lower order aberrations arising therein, thus for example spheres, cylinders and axis values, a specification cannot be provided herein, such that a lower order aberration may be introduced for the benefit of regularization, which may be compensated for by a separate correction, for example by spectacles or a refraction correction, after the treatment.

For compensating for the geometric irregularities, the treatment profile may for example include plane-parallel volumes to ablate the irregularities. In particular, the treatment may also extend beyond the optical zone, for example into a transition zone or even into an epithelial profile outside of the stromal total ablation zone. In particular, the largest allowed optical zone, which may be preset by wavefront treatments, may be used.

The optimization function may be a cost function, wherein an ablation depth or an ablation volume, which is planned in the treatment and removal of the geometric irregularities, is used as the term of the tissue removal. In particular, it may be optimized by iterative methods until the optimization function is in an optimization range, for example in a range of 10% around an optimum value.

If the treatment profile has been found by the optimization function, the corresponding treatment profile may be provided to the treatment apparatus, in particular to the ophthalmological laser, in the form of control data, wherein the laser may be subsequently controlled with the control data. In particular, the laser may be an ablation laser, by which corneal tissue is ablated, however, a photodisruptive laser may also be used, by which incisions are generated in the cornea and thus the geometric irregularities may be removed.

By the invention, the advantage arises that vision impairing effects of the geometric irregularities may be reduced, in particular higher order aberrations, wherein as much tissue as possible may be saved.

The invention also includes further embodiments, by which additional advantages arise.

An embodiment provides that the geometric irregularities are determined by a wavefront measurement of the eye, wherein a corneal ideal profile, which does not have geometric irregularities and higher order aberrations, is defined by a wavefront ideal profile, wherein the optimization function is optimized depending on the corneal ideal profile. This means that the predetermined examination data includes at least one wavefront measurement of the eye, wherein the treatment profile is determined with the aid of a wavefront ideal profile. The wavefront ideal profile may be a specification how the cornea is to look after the treatment, wherein the wavefront ideal profile thus results in a corneal ideal profile. In particular, it does not have geometric irregularities and/or higher order aberrations. Thus, an optimization may for example be performed by superposition of the present corneal profile with the corneal ideal profile in that the corneal ideal profile is for example changed along a common axis until the optimization function is in the optimization range.

A further embodiment provides that the geometric irregularities are provided by a corneal profile from a tomography and/or topography measurement of a cornea of the eye, wherein a corneal ideal profile is defined, which does not have geometric irregularities and higher order aberrations, wherein the optimization function is optimized depending on the corneal ideal profile. This means that, in this embodiment, the examination data is determined by a tomography and/or topography measurement and the geometric irregularities are present as the corneal profile. Herein, one may define a corneal ideal profile, which may for example be superimposed with a common axis of the corneal profile and is varied or shifted along the axis until the optimization function is in the optimization range.

In the following embodiments, it may be provided that the used corneal profile and the corneal ideal profile are provided either by the wavefront measurement or from the tomography and/or topography measurement.

An embodiment provides that, for optimizing the optimization function, the corneal ideal profile is superimposed with the corneal profile and shifted along a vertical axis of the corneal profile until the optimization function reaches the optimization range, wherein only those areas of the corneal profile, which are above the treatment profile in the direction of the vertical axis, are set to be removed. In other words, by shifting the corneal ideal profile on the vertical axis, a position may be found, in which the geometric irregularities, which are above the corneal ideal profile, are cut without therein too much tissue being removed.

A further embodiment provides that the corneal ideal profile is superimposed with the corneal profile and tilted against a vertical axis of the corneal profile until the optimization function reaches the optimization range, wherein only those areas of the corneal profile, which are above the treatment profile in the direction of the vertical axis, are set to be removed. Herein, it may be accepted that the tilted corneal ideal profile generates a cone or a prism, which is used as the treatment profile, wherein the prism may generate lower order aberrations. However, these lower order aberrations may be compensated for by a spectacle correction.

A further embodiment provides that the corneal ideal profile is superimposed with the corneal profile and a curvature of the corneal ideal profile is changed until the optimization function reaches the optimization range, wherein only those areas of the corneal profile, which are above the treatment profile, are set to be removed. This means that a refraction profile for generating a curvature change of the cornea may be determined by the change of the curvature of the corneal ideal profile, wherein multiple irregularities may be additionally at least partially compensated for and thus minimized by the curvature change. In this embodiment, a refraction may be introduced by the curvature change, in particular lower order aberrations, but which may for example be again compensated for by a spectacle correction. Herein, the aim may be that the higher order aberrations of the geometric irregularities may be reduced by the introduction of the lower order aberrations. For example, it may be provided that only a low refraction or lower order aberrations are introduced such that the tissue removal is in particular minimized. The refraction values of sphere, cylinder and axis may in particular be freely selectable and independent of the patient. This embodiment serves for obtaining symmetry, wherein either a global curvature change of the corneal ideal profile or a curvature change in multiple planes, for example two different curvatures, which are perpendicular to each other, may be generated to generate a target asphericity.

A further form of configuration provides that the irregularities include a keratoconus, a keratoglobus, a pellucid marginal degeneration of the cornea, a herpes simplex keratitis and/or improper treatments of the cornea. This means that a usual optical error, such as for example a lower order aberration, in particular myopia or hyperopia, is not considered herein as a geometric irregularity, but unnatural elevations and/or depressions and/or scar tissue in the eye are considered geometric irregularities.

A further embodiment provides that the examination data includes a wavefront measurement and/or a tomography measurement and/or a topography measurement of the cornea, by which the geometric irregularities of the cornea are determined. For example, the wavefront measurements may include corneal or ocular wavefronts, and the measurements may be aligned to a relevant point, for example centered on a pupil, decentered on a pathological point and/or on a corneal apex, a thinnest location of the cornea and/or on a point of symmetry.

A further form of configuration provides that a maximum depth of the tissue removal is limited to below 50 μm. In particular, this may be provided in case of a maximized optical zone. In particular, the 50 μm may be provided below the epithelial tissue, that is in a stroma of the cornea. If the optimization function cannot be optimized up to the optimization range with maximized optical zone and a variation of the higher and lower order aberrations, the optical zone may be adapted, wherein the higher and lower order aberrations may for example be again adapted with a smaller optical zone to optimize the optimization function.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect of the invention relates to a control device, which is formed to perform the steps of at least one embodiment of the previously described method. Thereto, the control device may comprise a computing unit for electronic data processing such as for example a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser may be formed to at least partially separate a predefined corneal volume with predefined interfaces of a human or animal eye by optical breakdown, in particular at least partially separate it by photodisruption, and/or to ablate corneal layers by (photo) ablation.

In a further advantageous embodiment of the treatment apparatus according to the invention, the laser may be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown may be spatially narrowly limited such that a high incision accuracy is allowed in the generation of the interfaces. In particular, the range between 700 nm and 780 nm may also be selected as the wavelength range.

In a further advantageous embodiment of the treatment apparatus according to the invention, the control device may comprise at least one storage device for at least temporary storage of at least one control dataset, wherein the control dataset or datasets include(s) control data for positioning and/or for focusing individual laser pulses in the cornea; and may comprise at least one beam device for beam guidance and/or beam shaping and/or beam deflection and/or beam focusing of a laser beam of the laser.

A further aspect of the invention relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster may access the computer-readable medium and read out the content thereof. The storage medium is for example formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the developments of another one of the aspects of the invention. Thus, the features of the embodiments of the invention may be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, configurations are also to be regarded as encompassed and disclosed by the invention, which are not explicitly shown or explained in the figures, but arise from and may be generated by separate feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram for a method according to an exemplary embodiment.

FIG. 3 depicts a schematic representation for determining an optimized treatment profile according to an exemplary configuration.

FIG. 4 depicts a schematic representation for determining an optimized treatment profile according to a further exemplary configuration.

FIG. 5 depicts a schematic representation for determining an optimized treatment profile according to a further exemplary configuration.

### DETAILED DESCRIPTION

In the figures, identical or functionally identical elements are provided with the same reference characters.

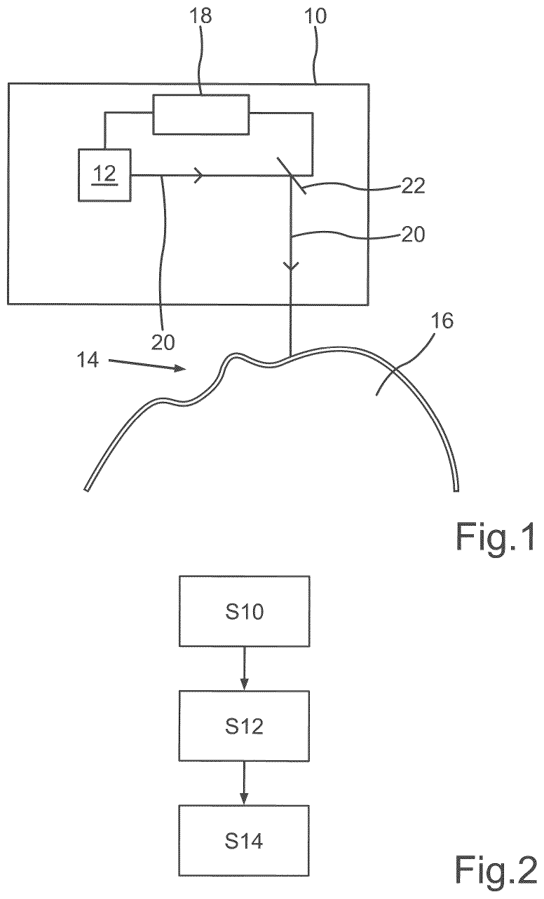

FIG. 1 shows a schematic representation of a treatment apparatus 10 with an ophthalmological laser 12 for reducing geometric irregularities 14 from a human or animal cornea 16 by photodisruption and/or ablation. For example, the geometric irregularities 14 of the cornea 16 may be caused by inflammations, improper treatments or degenerations of the cornea 16. Therein, the geometric irregularities 14 may locally change a curvature of the cornea 16, which generates higher order visual disorders, in particular higher order aberrations. A treatment profile for correcting the geometric irregularities 14 may be provided by a control device 18, in particular in the form of control data, such that the laser 12 emits pulsed laser pulses in a pattern predefined by the control data into the cornea 16 of the eye. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22 such as for example a rotation scanner, to remove the geometric irregularities 14. The beam deflection device 22 may also be controlled by the control device 18.

The illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

For determining control data, which comprises the treatment profile for reducing or removing the geometric irregularities 14, the control device 18 may for example perform the method shown in FIG. 2.

In FIG. 2, a schematic method diagram for providing control data for the ophthalmological laser 12 of the treatment apparatus 10 is illustrated, wherein a reduction of the geometric irregularities 14 may be achieved with the method. The method steps described below may in particular be performed by the control device 18 of the treatment apparatus 10 or by an external control device for planning the treatment.

In a step S10, the geometric irregularities 14 of the eye may be determined from predetermined examination data, wherein the geometric irregularities 14 generate higher order aberrations. For determining the examination data, wavefront measurements and/or tomography measurements and/or topography measurements of the cornea may for example be performed.

In a step S12, a treatment profile, which has a preset optical zone, may be determined, wherein an optimization function may be optimized up to an optimization range thereto, in particular up to an optimization value. In particular, the optimization function may comprise a term for reducing the higher order aberrations and an opposing tissue removal term, wherein the optimization function may for example be a cost function, which may be iterated to a maximized reduction of the higher order aberrations and a minimized tissue removal. Hereto, a corneal ideal profile 26 may for example be defined, which is varied with respect to a measured corneal profile 24, until the higher order aberrations and the tissue removal are minimized. Herein, the corneal ideal profile 26 may be directly preset or be derived from a wavefront ideal profile.

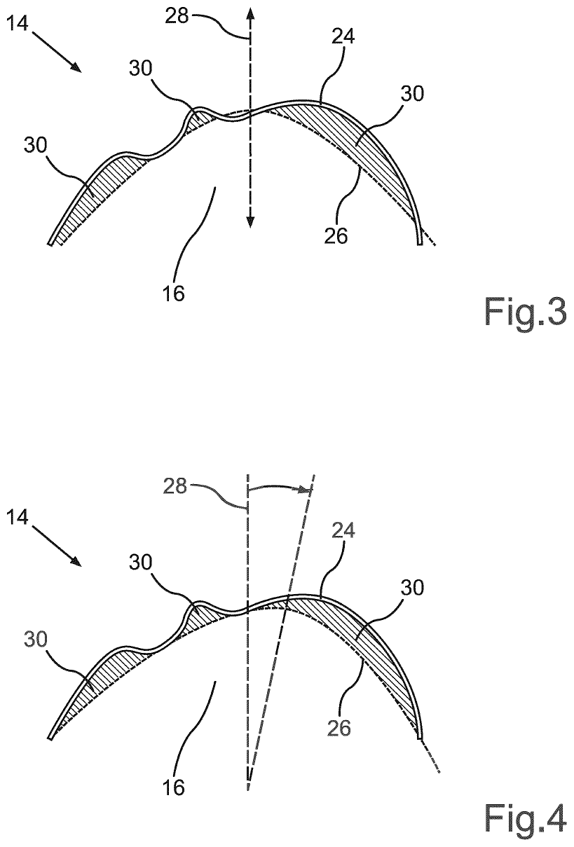

A configuration for determining the treatment profile by a corneal ideal profile 26 for optimizing the optimization function is for example illustrated in FIG. 3. Herein, the corneal profile 24 of the cornea 16 may be ascertained from wavefront measurements or tomography and/or topography measurements and the corneal ideal profile 26 may be preset. Therein, an optical zone (not shown) may radially limit the treatment area or the corneal ideal profile 26, wherein a maximized optical zone, for example of a scotopic pupil, may be preset. Subsequently, the corneal ideal profile 26 and the corneal profile 24 may be superimposed, for example at a preset point or a preset axis, and be shifted against each other along a vertical axis 28 until the optimization function reaches the optimization range. If both the higher order aberrations and the tissue removal are minimized, the position of the corneal ideal profile 26 may be set as the treatment profile, wherein only those areas 30, which are between the treatment profile or corneal ideal profile 26 and the corneal profile 24 in the direction of the vertical axis 28, are set to be removed. This means that the areas 30 may be removed for reducing the geometric irregularities 14.

In FIG. 4, a further configuration for determining a treatment profile by the corneal ideal profile 26 is schematically illustrated. In this configuration, the corneal profile 24 of the cornea 16 may be again predetermined from the examination data and the corneal ideal profile 26 may be preset. For optimizing the optimization function, in this configuration, the corneal ideal profile 26 may be tilted around the vertical axis 28, in particular until the optimization function reaches the optimization range. Then, the areas 30, which are between the treatment profile or tilted corneal ideal profile 26 and the corneal profile 24, may be set to be removed. In this configuration, it may occur that an additional lower order aberration is generated by the tilted corneal ideal profile 26, but which is accepted in this configuration, since lower order aberrations may be compensated for by spectacle corrections.

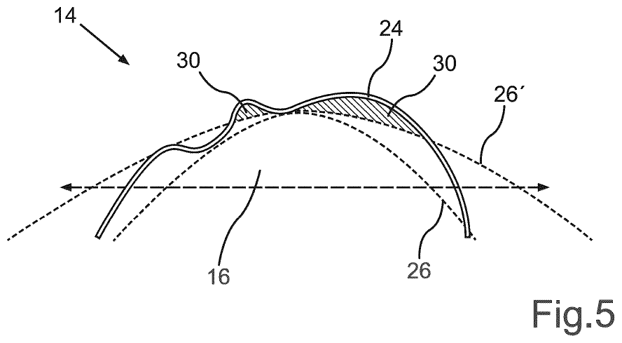

A further configuration for determining the treatment profile by the corneal ideal profile 26 is illustrated in FIG. 5. After the corneal profile 24 has been ascertained and the corneal ideal profile 26 has been preset, in this configuration, a curvature of the corneal ideal profile 26 may be changed after superposition of the corneal ideal profile 26 with the corneal profile 24, until the optimization function reaches the optimization range. In this example, the corneal ideal profile 26 may be changed to an optimized corneal ideal profile 26′ with lower radius of curvature to reach the optimization range. In particular, different planes, i.e. different radii of curvature of the corneal ideal profile 26, may be adapted separately from each other in order that the optimization function reaches the optimization range. Subsequently, the areas 30, which are between the optimized corneal ideal profile 26′ and the corneal profile 24, may again be set for removal.

The configurations, which are described in FIGS. 3, 4 and 5, may be combined with each other such that the treatment profile, with which the greatest reduction of the higher order aberrations and the lowest tissue removal may be provided, may be ascertained by the optimized optimization function. Furthermore, it may be provided that the tissue removal is limited to a preset value, for example to a depth of less than 50 μm.

Finally, control data may be provided in a step S14, which includes at least the ascertained treatment profile.

1. A method for providing control data for an ophthalmological laser of a treatment apparatus for reducing geometric irregularities of an eye, wherein the method comprises the following steps performed by a control device:
determining geometric irregularities of the eye from predetermined examination data, which generate higher order aberrations;
determining a treatment profile with a preset optical zone depending on the geometric irregularities, wherein an optimization function, which includes a term for reducing the higher order aberrations and an opposing tissue removal term, is optimized up to an optimization range for determining the treatment profile; and
providing the control data, which includes the treatment profile.

2. The method according to claim 1, wherein the geometric irregularities are determined by a wavefront measurement of the eye, wherein a corneal ideal profile, which does not have geometric irregularities and higher order aberrations, is defined by a wavefront ideal profile, wherein the optimization function is optimized depending on the corneal ideal profile.

3. The method according to claim 1, wherein the geometric irregularities are provided by a corneal profile from a tomography and/or topography measurement of a cornea of the eye, wherein a corneal ideal profile is defined, which does not have geometric irregularities and higher order aberrations, wherein the optimization function is optimized depending on the corneal ideal profile.

4. The method according to claim 3, wherein the corneal ideal profile is superimposed with the corneal profile and shifted along a vertical axis of the corneal profile for optimizing the optimization function, until the optimization function reaches the optimization range, wherein only those areas of the corneal profile, which are above the treatment profile in a direction of the vertical axis, are set to be removed.

5. The method according to claim 3, wherein the corneal ideal profile is superimposed with the corneal profile and tilted against a vertical axis of the corneal profile until the optimization function reaches the optimization range, wherein only those areas of the corneal profile, which are above the treatment profile in a direction of the vertical axis, are set to be removed.

6. The method according to claim 3, wherein the corneal ideal profile is superimposed with the corneal profile and a curvature of the corneal ideal profile is changed until the optimization function reaches the optimization range, wherein only those areas of the corneal profile, which are above the treatment profile, are set to be removed.

7. The method according to claim 1, wherein the geometric irregularities include a keratoconus, a keratoglobus, a pellucid marginal degeneration of a cornea of the eye, a herpes simplex keratitis and/or improper treatments of the cornea.

8. The method according to claim 1, wherein the predetermined examination data includes a wavefront measurement and/or a tomography measurement and/or a topography measurement of a cornea, by which the geometric irregularities of the eye are determined.

9. The method according to claim 1, wherein a maximum depth of tissue to be removed is limited to below 50 μm.

10. A control device which is configured to perform a method according to claim 1.

11. A treatment apparatus with at least one ophthalmological laser for removing a corneal volume of a human or animal eye by optical breakdown and at least one control device according to claim 10.

12. (canceled)

13. A non-transitory, computer-readable medium for storing a computer program the computer program comprising commands which cause a treatment apparatus to execute the method according to claim 1.
